(19) United States
(12) Patent Application Publication
(10) Pub. No.: US 2007/0171364 A1
(43) Pub. Date: Jul. 26, 2007

(54) Method of relieving computer vision syndrome

(76) Inventor: Steven M. Beresford, Camas, WA (US)

Correspondence Address: STEVEN M. BERESFORD, 19300 NE 6th STREET, CAMAS, WA, 98607, US

(21) Appl. No.: 11/335,885

(22) Filed: Jan. 20, 2006

(51) Int. Cl. A61B 3/00

(52) U.S. Cl. 351/203

(57) ABSTRACT

This invention comprises a method that enables a computer user to relieve or avoid computer vision syndrome by means of eye exercises and stress reduction techniques. One aspect of the invention consists of a computer program that displays eye exercises on the computer screen after a predetermined time interval or after a predetermined number of keystrokes. Another aspect of the invention consists of a computer program that displays a message on the computer screen after a predetermined time interval or after a predetermined number of keystrokes or during an asynchronous time break, reminding the user to perform stress reduction techniques. Another aspect of the invention is a computer program that displays information on the computer screen about computer ergonomics, the visual system, nearpoint stress, and computer vision syndrome.

### BACKGROUND OF THE INVENTION

Our ancestors were hunters and warriors whose survival depended on accurately seeing enemies and animals far away. For this reason, the visual system evolved for good distance vision; specifically, gazing at the horizon for predators or prey. Modern civilization, especially computer use, places enormous demands on the visual system that are diametrically opposed to its natural function and abilities.

Many computer users spend long periods of time doing near work, which typically involves visually tracking, decoding, and processing masses of printed material. This causes “nearpoint stress”, which is the result of sustained convergence, near focusing, and repetitive eye movements that are not part of the visual system's natural function.

Nearpoint stress gives rise to the physiological condition known as “computer vision syndrome” (CVS), which consists of one or more of the following symptoms: eyestrain; dry, tired, sore eyes; itchy, gritty sensations in and around the eyes; eyelid tics or spasms; blurred or double vision; loss of distance vision; headaches, fatigue, dizziness; sensations of being “stressed out”, overwhelmed, or disorientated.

Treatment procedures for CVS include magnifying glasses, software programs and screen overlays that magnify the text, and eye drops or medication to relieve the discomfort. None of these procedures addresses the underlying problem, which is that the visual system does not have the intrinsic strength or ability to neutralize and overcome the nearpoint stress. The logical way of addressing the underlying problem is to strengthen the eyes with exercises and practice stress reduction techniques when using the computer.

### CROSS REFERENCE TO RELATED APPLICATIONS

The use of eye exercises and stress reduction techniques during rest breaks to relieve CVS was first proposed by the author in his 1988 monograph “How to Stop Computer Stress or Eyestrain” (attached). In addition to the monograph, an audio cassette tape provided two sequences of exercises and a reminder card (attached) advising the user to take rest breaks and use stress reduction techniques.

The author then proposed the use of eye exercises and rest breaks to relieve CVS to Microsoft Corp. in 1992 and in 1996. Those proposals were rejected. The use of eye exercises and rest breaks to relieve CVS was also proposed in the author's book “Improve Your Vision Without Glasses or Contact Lenses” (Drs. Steven M. Beresford, David M. Muris, Merrill J. Allen, Francis A. Young; Simon & Schuster, 1996), and then in U.S. Provisional Patent Application 60/171,513 and U.S. Patent Application 60/174,881, which were filed on Dec. 22, 1999 and Jan. 7, 2000 respectively. Those patent applications attempted to patent the exercise sequences but were denied on the grounds that the exercise sequences were in the public domain. No attempt was made to patent the software architecture or the novel features of the exercises, which are the subjects of the present application.

Related US patents include U.S. Pat. No. 6,075,525 (Hsieh, 2000); U.S. Pat. No. 5,888,173 (Singhal, 1999); U.S. Pat. No. 6,364,485 (Fateh, 2002); U.S. Pat. No. 5,305,238 (Starr, 1994).

### SUMMARY OF THE INVENTION

The invention consists of a computer program, typically in the form of a CD-ROM or Internet download, comprising the following elements:

1) Programming instructions that display information on the computer screen regarding computer ergonomics including screen positioning; glare reduction; brightness and contrast adjustment; chair, keyboard, mouse, and document holder positioning; desktop organization and positioning.

2) Programming instructions that display information on the computer screen about the visual system, nearpoint stress, and CVS.

3) Programming instructions that display descriptions on the computer screen of the exercises and stress reduction techniques.

4) Programming instructions that display sequences of exercises on the computer screen at predetermined intervals.

5) Programming instructions that display reminder messages on the computer screen at predetermined intervals to perform the stress reduction techniques.

### DESCRIPTION OF THE EXERCISES

The exercises fall into two major groups, which are described below. The term “distant object” means an object that is more than a few feet away from the computer screen.

GROUP A. Relaxation Exercises.

The following exercises relax and revitalize the eyes, thereby reducing the impact of nearpoint stress on the visual system:

1) Slow Blinking. This exercise involves breathing deeply and looking at a small detail on a distant object when inhaling. When exhaling, the eyes are kept closed and relaxed.

2) Squeeze Blinking. This exercise involves breathing deeply and looking at a small detail on a distant object when inhaling. When exhaling, the eyelids are squeezed tightly shut.

3) Edging. This exercise involves breathing deeply and running the gaze along the edges of a distant object,

4) Centering. This exercise involves breathing deeply and looking at the smallest visible detail on a distant object.

5) Acupressure. This exercises consists of four variations and involves massaging the acupressure points surrounding the eyes. A preferred embodiment of the exercise is to coordinate the massage strokes with deep breathing.

GROUP B: Strengthening Exercises.

The following exercises increase ocular coordination and focusing ability, thereby enabling the eyes to function more comfortably and efficiently when looking at a computer screen:

1) Pumping. This exercise involves changing focus every few seconds between a near object such as a thumb or pencil held a few inches in front of the eyes and a distant object, briefly looking at a small detail on each object. A preferred embodiment of the exercise is to coordinate the change of focus with deep breathing. For example, look at the near object when inhaling and at the distant object when exhaling.

2) Tromboning. This exercise involves looking at a small detail on an object such as a thumb or pencil while slowly moving it back and forth between arm's length and the tip of the nose. A preferred embodiment of the exercise is to coordinate the arm movement with deep breathing. For example, bring the object toward the nose when inhaling and take it to arm's length when exhaling.

3) Clock Rotations. This exercise involves moving the eyes in the direction of each number on the face of an imaginary clock, then back to the center of the clock. A preferred embodiment of the exercise is to coordinate the eye movements with deep breathing. For example, look at the center of the clock when inhaling and at the number when exhaling.

4) Eye Rolls. This exercise involves slowly moving the eyes in partial or complete circles with the eye muscles fully stretched. A preferred embodiment of the exercise is to coordinate the eye movements with deep breathing. For example, move the eyes in one direction when inhaling and in the opposite direction when exhaling.

Supplementary Exercises. In addition to the exercises described above and miscellaneous variations thereof, other exercises can be used including but not limited to the following:

1) Light Therapy. This exercise involves sitting in front of an incandescent, fluorescent, or halogen light with the eyes closed.

2) Palming. This exercise involves covering the closed eyes with the palms of the hands so that no light enters the eyes.

3) Nose Fusion. This exercise involves looking back and forth between the tip of the nose and a distant object. A preferred embodiment of the exercise is to coordinate the movements with breathing. For example, look at the nose when inhaling and at a distant object when exhaling.

4) Thumb Pursuits. This exercise involves looking at a small detail on the thumb of one hand while slowly moving the thumb in front of the face in different directions and distances. A preferred embodiment of the exercise is to coordinate the arm movements with breathing. For example, bring the thumb toward the face when inhaling and take it away from the face when exhaling.

5) Fast Blinking. This exercise involves breathing deeply and looking at a small detail on a distant object when inhaling. When exhaling, the eyes are opened and closed as rapidly as possible.

Novel Elements. Although basic forms of some of the exercises are in the public domain, the author has created the following novel elements:

1) Slow Blinking, Squeeze Blinking, and Fast Blinking are entirely new exercises.

2) In prior art, the exercises were not coordinated with breathing. The author has introduced coordination with breathing as a novel element in the following exercises: Slow Blinking, Squeeze Blinking, Acupressure, Pumping, Tromboning, Clock Rotations, Eye Rolls, Nose Fusion, Thumb Pursuits, Fast Blinking.

3) In prior art, the exercises did not involve looking at small details. The author has introduced looking at small details as a novel element in the following exercises: Slow Blinking, Squeeze Blinking, Pumping, Tromboning, Centering, Nose Fusion, Thumb Pursuits, Fast Blinking.

Description of the Software Architecture

The software architecture of a computer program or computing system is defined as the structure or structures of the system, which comprise software elements, the externally visible properties of those elements, and the relationships between them.

This invention consists of the following software architecture, where the term “descriptions” includes text and audio output, and the term “illustrations” includes drawings, photographs, videos, and animations.

1) Ergonomic Information. Programming instructions that display information on the computer screen about computer ergonomics, including descriptions, illustrations, and effects, which enable the user to set up the computer for maximum comfort and efficiency.

2) CVS Information. Programming instructions that display information on the computer screen about the visual system, nearpoint stress, and CVS including descriptions, illustrations, effects, and symptoms.

3) Exercise Information. Programming instructions that display information on the computer screen about the exercises including descriptions, illustrations, and effects.

4) Stress Reduction Information. Programming instructions that display information on the computer screen about the stress reduction techniques including descriptions, illustrations, and effects.

5) Exercise Sequences. Programming instructions that display sequences of exercises on the computer screen at predetermined time intervals or after a predetermined number of keystrokes.

6) Reminder Messages. Programming instructions that display reminder messages on the computer screen to practice the stress reduction techniques at predetermined time intervals, or after a predetermined number of keystrokes, or during asynchronous time breaks.

Description of the Exercise Sequences

As a result of his research, the author has determined that certain groups and sequences of exercises produce the best results. People who already suffer from CVS should use relaxation exercises until they become asymptomatic, then they should use a combination of relaxation exercises and strengthening exercises. In contrast, people who do not suffer from CVS but want to avoid it should use strengthening exercises.

A preferred embodiment of exercise groups and sequences is:

Relaxation (A): Slow Blinking, Edging, Squeeze Blinking, Centering.

Relaxation (B): All four variations of the Acupressure exercises.

Strengthening: Pumping, Clock Rotations, Tromboning, Eye Rolls.

These sequences are practiced on a regular basis when using the computer. For example, a different sequence may display on the screen every hour with each exercise lasting for one minute. In a preferred embodiment, a video of a human figure or animated figure displays on the screen and performs the exercises to the accompaniment of music and instructions, encouraging the user to imitate the displayed movements.

In another embodiment, a clock or other timing device displays on the screen for the duration of the exercise together with written, verbal, or graphic instructions encouraging the user to practice the exercise.

The exercise sequences can be programmed to display on the screen at predetermined time intervals or after a predetermined number of keystrokes. The exercise sequences terminate after a predetermined time interval, or when the user operates the mouse or keyboard.

The preferred embodiment of the invention includes preset exercise sequences, durations, time intervals, or keystroke intervals, with the exercise sequences displayed in rotation. For example, after an hour the Relaxation (A) sequence is displayed with each exercise lasting for one minute. An hour later, the Relaxation (B) sequence is displayed. An hour later, the Strengthening sequence is displayed. In addition to the presets, the user can modify the sequences, durations, time intervals, or keystroke intervals to meet his or her own preferences. For example, a person suffering from sever headaches may choose to rotate the Relaxation sequences, with Relaxation (A) and Relaxation (B) following each other every 20 minutes. In another example, a person who is fully asymptomatic may decide to do only the Strengthening sequence and have it display every two hours. In another example, the user may decide to create a new, customized sequence such as Pumping, Slow Blinking, Centering, Acupressure, Tromboning, and Squeeze Blinking, and have the sequence display every 90 minutes.

Description of the Reminder Messages

Another aspect of the invention is the ability to display reminder messages on the screen at predetermined intervals, instructing the user to perform a specific stress reduction technique, which typically consists of one repetition of one of the Relaxation or Strengthening exercises. Examples of reminder messages include:
Now Do A Slow Blink!
Now Do Some Edging!
Now Do Some Pumping!

The reminder messages may be static, flashing, or animated, and may contain an audio prompt or an illustration or description of the stress reduction technique. A preferred embodiment of the invention includes a preset sequence of messages with preset sizes, colors, fonts, positions, durations, time intervals, and keystroke intervals, with the messages displayed in rotation. For example, after 10 minutes the first message is displayed for 10 seconds, then 10 minutes later the second message is displayed for 10 seconds, etc.

In addition to the presets, the user can adjust the parameters to meet his or her own preferences. For example, a user suffering from headaches may decide to display a different message every 3 minutes. In another example, the person may decide to display a single message every 15 minutes. In another example, the user may decide to create new messages or new sequences of messages.

In addition to adjusting the messages to display at predetermined time intervals or after a predetermined number of keystrokes, the messages can be made to display during asynchronous time breaks. For example, a message can be displayed when the person is bringing up a file from the computer's memory, or saving a file into the computer's memory, or waiting for a program to execute a task such as going online. The message terminates after a predetermined time interval, or when the user operates the mouse or keyboard.

The invention's reminder message function may be used together with or independently of the exercise sequence function described previously. For example, a user may decide not to do any of the exercise sequences but simply display the reminder messages at 3 minute intervals.

Description of the Computer Medium

“Computer” means any computing device with a video display terminal, which is referred to herein as a “screen”. The invention is embodied in a “computer readable medium”, which means any device used to store data accessible by a computer such as a magnetic hard disc, a floppy disc, a video disc, a CD-ROM, a DVD, a memory chip, and a carrier wave used to transmit and receive data in accessing a network. “Computer program” refers to a set of commands to operate a computer such as programmed logic and instructions. A “network” refers to a number of computers and associated devices connected together by means of communication facilities such as cables, telephone lines, fiber optics, and electromagnetic waves. Examples of a network include the Internet, a local area network (LAN), and a wide area network (WAN). In yet another embodiment of the invention, the program may be directly integrated into a video display terminal as a function of the device driver or the video display terminal hardware.

As mentioned above, the invention can be embodied in a carrier wave used to access a network. For example, a user may access an Internet website via a wireless network then download the program and install it on his or her computer.

It is further contemplated that some aspects of the invention will provide sophisticated sequences that are tailored toward individual users. For example, a user may enter subjective data such as current symptoms. Based upon such data, the system will select or recommend specific exercise sequences together with appropriate durations and/or time intervals, or will select or recommend specific reminder messages together with appropriate time intervals and/or keystroke intervals.

Although only a few embodiments of the present invention have been described, it should be understood that the present invention may be embodied in many other specific forms without departing from the spirit or scope of the invention. The present examples are to be considered as illustrative and not restrictive, and the invention is not to be limited to the details given herein, but may be modified within the scope of the appended claims.

What is claimed is:

1) A method of relieving computer vision syndrome (CVS), comprising a computer program that embodies a set of exercises and stress reduction techniques that strengthen and relax the eyes.

2) A method as recited in claim 1) consisting of a computer program with the following software architecture:
a) Programming instructions that display information on the computer screen about computer ergonomics, including descriptions, illustrations, and effects.
b) Programming instructions that display information on the computer screen about the visual system, nearpoint stress, and CVS including descriptions, illustrations, effects, and symptoms.
c) Programming instructions that display information on the computer screen about the exercises including descriptions, illustrations, and effects.
d) Programming instructions that display information on the computer screen about the stress reduction techniques including descriptions, illustrations, and effects.
e) Programming instructions that display specific exercises or sequences of exercises on the computer screen after a predetermined time interval or after a predetermined number of keystrokes.
f) Programming instructions that display messages, which may include illustrations and descriptions of the stress reduction techniques, on the computer screen after a predetermined time interval or after a predetermined number of keystrokes or during an asynchronous time break.

3) A method of exercises as recited in claim 1), consisting of but not limited to the following exercises: Slow Blinking, Squeeze Blinking, Edging, Centering, Acupressure, Pumping, Tromboning, Clock Rotations, Eye Rolls, Light Therapy, Palming, Nose Fusion, Thumb Pursuits, and Fast Blinking.

4) A method of stress reduction techniques as recited in claim 1), where each stress reduction technique consists of a single repetition of an exercise selected from those recited in claim 3).

5) A method of exercises as recited in claim 1), in which the following exercises are new and original: Slow Blinking, Squeeze Blinking, and Fast Blinking.

6) A method of exercises as recited in claim 1), in which the following exercises are performed in coordination with breathing: Slow Blinking, Squeeze Blinking, Edging, Centering, Acupressure, Pumping, Tromboning, Clock Rotations, Eye Rolls, Nose Fusion, Thumb Pursuits, and Fast Blinking.

7) A method of exercises as recited in claim 1), in which the following exercises are performed in conjunction with looking at a detail on a target object: Slow Blinking, Squeeze Blinking, Centering, Pumping, Tromboning, Nose Fusion, and Fast Blinking.

8) A method of exercises as recited in claim 1), in which the exercises are displayed on a computer screen in a predetermined sequence or configuration or in a sequence or configuration that can be determined by the user according to his or her preferences.

9) A method of exercises as recited in claim 1), in which the exercise sequence displayed on the computer screen terminates after a predetermined time interval or when the user operates the computer's mouse or keyboard.

10) A method of stress reduction techniques as recited in claim 1), in which reminder messages to practice stress reduction techniques are displayed on the computer screen after a predetermined time interval or after a predetermined number of keystrokes or during an asynchronous time break, in a predetermined sequence and configuration or in a sequence and configuration that can be determined by the user according to his or her preferences.

11) A method of stress reduction techniques as recited in claim 1), in which the reminder message displayed on the computer screen terminates after a predetermined time interval or when the user operates the computer's mouse or keyboard.

12) A method as recited in claim 2), in which the user can enter data into the program regarding current symptoms, whereupon the system will recommend or select specific exercise sequences together with appropriate durations or time intervals, or will recommend or select specific reminder messages together with appropriate time intervals or number of keystrokes.
